(19) United States
(12) Patent Application Publication
(10) Pub. No.: US 2026/0072031 A1
(43) Pub. Date: Mar. 12, 2026

(54) APPLICATION OF REAGENT FOR QUANTITATIVE DETECTION OF PYROGLUTAMYL AMINOPEPTIDASE IN THE DIAGNOSIS OF RHEUMATOID ARTHRITIS

(71) Applicants: Shandong First Medical University & Shandong Academy of Medical Sciences, Jinan (CN); Shandong Sky Grand Bio-pharmaceutical Co., Ltd., Heze (CN)

(72) Inventors: Renshuai ZHANG, Jinan (CN); Hongfei JIANG, Jinan (CN); Qiuyu GONG, Jinan (CN); Jiwei YU, Jinan (CN)

(21) Appl. No.: 19/394,503

(22) Filed: Nov. 19, 2025

(30) Foreign Application Priority Data
Apr. 11, 2025 (CN) 202510457426.8

(51) Int. Cl. G01N 33/573; C12Q 1/6883

(52) U.S. Cl. CPC G01N 33/573; C12Q 1/6883; C12Q 2600/118; C12Q 2600/158; G01N 2333/948; G01N 2800/102; G01N 2800/52

(57) ABSTRACT

This application provides an application of a reagent for quantitative detection of pyroglutamyl aminopeptidase in the diagnosis of rheumatoid arthritis (RA), where the reagent is a reagent for quantitative detection of pyroglutamyl aminopeptidase gene mRNA, a reagent for quantitative detection of pyroglutamyl aminopeptidase protein or a combination thereof. The quantitative detection is performed by enzyme-linked immunosorbent assay, Western blot, immunohistochemistry, a quantitative fluorescent polymerase chain reaction, mass spectrometry, or a combination thereof. A system for diagnosis of rheumatoid arthritis is also provided, including a sample collection module, a sample detection module and a data analysis module. The sample collection module is configured to collect a biological sample. The sample detection module is configured to quantitatively detect an expression level of pyroglutamyl aminopeptidase in vitro. The data analysis module is configured to compare a detection result with a diagnostic threshold, and generate a diagnosis report.

### CROSS-REFERENCE TO RELATED APPLICATIONS

This application claims the benefit of priority from Chinese Patent Application No. 202510457426.8, filed on Apr. 11, 2025. The content of the aforementioned application, including any intervening amendments thereto, is incorporated herein by reference in its entirety.

### TECHNICAL FIELD

This application relates to biological detection technologies, and more particularly to an application of a reagent for quantitative detection of pyroglutamyl aminopeptidase in the diagnosis of rheumatoid arthritis.

### BACKGROUND

Rheumatoid arthritis (RA) is a chronic, systemic and autoimmune disease characterized by synovial inflammation and progressive destruction of joint structures, potentially leading to joint deformity and loss of function. The pathogenesis of the RA is complex and closely related to genetic susceptibility, environmental factors, and dysregulated immune responses. The key pathological features of the RA include synovial hyperplasia, neovascularization, and extensive infiltration of immune cells, accompanied by excessive release of inflammatory factors. Early diagnosis and intervention are critical for delaying disease progression and improving life quality of patient. However, due to the diverse clinical manifestations and the lack of a single specific diagnostic criterion for RA, the diagnosis and progression assessment of the RA still have huge challenges.

At present, the clinical diagnosis of the RA primarily relies on comprehensive medical history, imaging examinations, and laboratory tests, and common detection methods are described as follows. (1) Serological biomarkers: anti-cyclic citrullinated peptide antibody (CCP) has relatively high specificity and sensitivity, but some patients test negative. (2) Rheumatoid factors (RF): RF is the earliest biomarkers used in RA diagnosis, but has low specificity and is easily disturbed by other diseases or physiological conditions. (3) Inflammatory indicators: C-reactive protein (CRP) and erythrocyte sedimentation rate (ESR) are valuable for reflecting inflammatory activity but lack specificity for RA. (4) Imaging examination: joint destruction is detected through X-ray, ultrasound, or magnetic resonance imaging (MRI), however, specific changes are often difficult to detect in the early stage of the RA. Although the methods above play a significant role in diagnosis and monitoring of the RA, but still have many problems as follows. (1) Existing indicators are not suitable for the assessment of disease progression and prognosis conditions of the RA, resulting in failure in guiding medication or therapy. (2) Approximately 20-30% of the RA patients are seronegative, that is, the RA patients are diagnosed with negative RF and negative CCP, and defined medically as seronegative rheumatoid arthritis. Therefore, it is urgent to discover and develop novel diagnostic biomarkers to further improve diagnostic accuracy and guide disease stratification in RA.

RA and osteoarthritis (OA) are two common joint disorders with distinct pathological mechanisms and treatment strategies. RA is an autoimmune disease characterized by synovial inflammation, immune cell infiltration, and joint destruction, while OA is a degenerative condition primarily caused by articular cartilage degradation and osteophyte formation. Clinically, RA and OA may present overlapping symptoms, in early-stage, such as joint pain, stiffness, and functional impairment. When OA occurs on finger joints, it may be misdiagnosed as RA, which brings a challenge for accurate diagnosis. Therefore, it is urgent to develop a method for effectively distinguishing and diagnosing RA and OA.

### SUMMARY

In order to solve at least one defects of the prior art, this application provides a rheumatoid arthritis diagnostic product based on reagents for detection of pyroglutamyl aminopeptidase.

Technical solutions of this application are described as follows.

In a first aspect, this application provides an application of a reagent for quantitative detection of pyroglutamyl aminopeptidase in the preparation of a product for diagnosis or prognostic assessment of rheumatoid arthritis.

In an embodiment, the reagent for quantitative detection of pyroglutamyl aminopeptidase is a reagent for quantitative detection of pyroglutamyl aminopeptidase gene mRNA, a reagent for quantitative detection of pyroglutamyl aminopeptidase protein or a combination thereof.

In an embodiment, the rheumatoid arthritis is selected from the group consisting of rheumatoid factor-positive (RF-positive) rheumatoid arthritis, anti-cyclic citrullinated peptide antibody-positive (CCP-positive) rheumatoid arthritis, RF-negative and CCP-negative rheumatoid arthritis, and a combination thereof.

In an embodiment, the quantitative detection is performed by enzyme-linked immunosorbent assay (ELISA), Western blot, immunohistochemistry, a quantitative fluorescent polymerase chain reaction (PCR), mass spectrometry, or a combination thereof.

In an embodiment, the product for diagnosis of rheumatoid arthritis or the product for prognostic assessment of rheumatoid arthritis is a kit, a chip, a test strip, a high-throughput sequencing platform or a combination thereof.

In an embodiment, the product for diagnosis of rheumatoid arthritis is adapted to diagnosis of rheumatoid arthritis in chronic joint diseases comprising rheumatoid arthritis and osteoarthritis, classification diagnosis of rheumatoid arthritis or a combination thereof.

In a second aspect, this application provides a detection system for diagnosis of rheumatoid arthritis, comprising:
a sample collection module;
a sample detection module; and
a data analysis module;
wherein the sample collection module is configured to collect a biological sample from a subject;
the sample detection module is configured to quantitatively detect an expression level of pyroglutamyl aminopeptidase in vitro; and
the data analysis module is configured to compare a detection result with a diagnostic threshold, and generate a diagnosis report.

In an embodiment, the biological sample is selected from the group consisting of whole blood, serum, plasma, tissue fluid, and a combination thereof.

In an embodiment, the diagnostic threshold is a cut-off value; and in an embodiment, the cut-off value is 57.28 ng/ml, that is, in a case that a subject has the expression level of pyroglutamyl aminopeptidase greater than or equal to 57.28 ng/ml, it can be preliminarily determined that the subject has RA.

In an embodiment, the data analysis module is configured to generate a comprehensive diagnosis report considering detection results of C-reactive protein (CRP), erythrocyte sedimentation rate (ESR), rheumatoid factors (RF) and anti-cyclic citrullinated peptide antibody (CCP).

In a third aspect, this application provides a product for diagnosis or prognostic assessment of rheumatoid arthritis, comprising: a reagent for detecting an expression level of pyroglutamyl aminopeptidase in a sample.

In an embodiment, the product for diagnosis or prognostic assessment of rheumatoid arthritis is selected from the group consisting of an enzyme-linked immunosorbent assay (ELISA) kit, a western blot kit, an immunohistochemistry staining kit, an immunochromatographic test strip and a combination thereof.

In a fourth aspect, this application provides a method for assessing a disease activity in rheumatoid arthritis, comprising detecting a change trend of an expression level of pyroglutamyl aminopeptidase in a patient before and after treatment, and detecting the disease activity.

In an embodiment, the method for assessing the disease activity in rheumatoid arthritis further comprises: assessing a disease severity considering inflammatory indicators involving CRP and ESR and imaging data.

In a fifth aspect, this application provides a method for formulating an individualized treatment plan for rheumatoid arthritis, comprising:

selecting appropriate drug, dosage, and treatment cycle based on a dynamic change of an expression level of pyroglutamyl aminopeptidase in a subject.

In an embodiment, the treatment strategy is optimized in combination with detection results of pyroglutamyl aminopeptidase and immune indicators of the subject including levels of interleukin-6 (IL-6) and tumor necrosis factor-α (TNF-α).

In a sixth aspect, this application provides an application of reagents for detection of pyroglutamyl aminopeptidase in the preparation of a product for differentiating rheumatoid arthritis from osteoarthritis.

In a seventh aspect, this application provides a method for diagnosis of rheumatoid arthritis in a subject in need thereof, comprising:
collecting a biological sample from the subject;
quantitatively detecting an expression level of pyroglutamyl aminopeptidase in the biological sample; and
comparing a detected expression level with a diagnosis threshold, and generating a diagnosis report based on comparison results, wherein if the detected expression level is equal to or larger than the diagnosis threshold, the subject is preliminarily identified as suffering from rheumatoid arthritis.

This application has the following beneficial effects.

(1) This application demonstrates that the level of pyroglutamyl aminopeptidase can be utilized for diagnosing seronegative rheumatoid arthritis, particularly in patients testing negative for both RF and CCP. Pyroglutamyl aminopeptidaseis utilized for diagnosing rheumatoid arthritis, which can address limitations of existing biomarkers and fills a gap in current RA diagnostic approaches.

(2) This application reveals that a linear correlation between the level of pyroglutamyl aminopeptidase and a RA progression. A dynamic assessment of the disease activity and a treatment response is realized through monitoring the level of pyroglutamyl aminopeptidase, providing a scientific basis for formulating individualized treatment plan.

(3) This application reveals that the level of pyroglutamyl aminopeptidase can be used to differentiate RA with similar clinical symptoms from OA, thereby improving diagnostic accuracy for RA.

In summary, pyroglutamyl aminopeptidase is utilized for diagnosing RA, which not only compensates for disadvantages of existing biomarkers, but also facilitates dynamic monitoring of disease activity and treatment response, while simultaneously enhancing differentiation between RA and OA. This application provides crucial support for RA diagnosis and precision medicine, and has significant clinical and commercial value.

### BRIEF DESCRIPTION OF THE DRAWINGS

FIG. 1 is a receiver operating characteristic (ROC) curve of pyroglutamyl aminopeptidase in serum of a rheumatoid arthritis (RA) subject.

FIG. 2 is a statistical graph for a differential expression of pyroglutamyl aminopeptidase detected at a protein level (***P<0.001).

FIG. 3 shows an expression level of pyroglutamyl aminopeptidase in a subject with seronegative rheumatoid arthritis (***P<0.001).

FIG. 4 shows a positive correlation between pyroglutamyl aminopeptidase and a clinical score of rheumatoid arthritis.

### DETAILED DESCRIPTION OF EMBODIMENTS

Some terms are described as follows.

Pyroglutamyl aminopeptidase (enzyme commission (EC): 3.4.19.3) catalyzes the removal of L-pyroglutamate from N-terminus of a certain peptide and protein. Pyroglutamyl aminopeptidase can be found across nearly all domains of life, including mammals, birds, fish, plants, protists, and fungi. Besides, pyroglutamyl aminopeptidase has high conservation and is widely distributed in human tissues, and its novel physiological functions continually being proposed in recent decades. In the prior art, due to pyroglutamyl aminopeptidase existed in small intestine and duodenum, it suggested to be involved in peptide and protein absorption in the mammalian digestive tract. In addition, due to its broad distribution in functionally diverse tissues, researchers have hypothesized that pyroglutamyl aminopeptidase may participate in intracellular peptide degradation and resynthesis. Pyroglutamyl aminopeptidase is also influence certain disease states, such as memory impairment, through modulating levels of free L-pyroglutamate.

Although pyroglutamyl aminopeptidase may be involved in regulation of certain diseases, its expression situation in rheumatoid arthritis (RA) remains unclear. More importantly, whether pyroglutamyl aminopeptidase can serve as a diagnostic indicator for RA lacks clear research evidence in the prior art.

This application reveals a linear correlation between a level of pyroglutamyl aminopeptidase and a RA progression, showing that disease progression and prognosis of rheumatoid arthritis can be assessed according to pyroglutamyl aminopeptidase, such assessment cannot be realized based on existing objective biomarkers, such as rheumatoid factors (RF) and anti-cyclic citrullinated peptide antibody (CCP). In addition, pyroglutamyl aminopeptidase can be used to diagnose seronegative rheumatoid arthritis, which supplements current diagnostic markers, such as RF and CCP, and addressing limitations of existing methodologies.

In order to better understand this application, the present disclosure will be further described below with reference to the specific embodiments, which are not intended to limit the present disclosure. Improvement and adjustment made by those of ordinary skill in the art without departing the spirit of the present disclosure, shall fall within the scope of this application defined by the appended claims.

In the following embodiments, test methods used herein are all conventional methods. Unless otherwise expressly specified and limited, materials and reagents used in the embodiments are conventional commercially available.

Examples 1-3 show data analysis of subject samples, where all RA cases were diagnosed according to 1987 Revised American College of Rheumatology Criteria for Rheumatoid Arthritis, which included assessment of the following indicators: “morning stiffness (lasting more than 1 hour)”, “joint symptoms (swelling in at least 3 joint areas)”, “rheumatoid nodules (subcutaneous nodules commonly found over areas susceptible to friction)”, “RF (rising in serum)”, and “X-ray (evidence of bone erosion)”. All cases of osteoarthritis patients met the diagnostic criteria for OA proposed by Altam, that is, the tests included: tissue hypertrophy, bony hypertrophy, bone friction sensation, and morning stiffness of at least two finger joints. All clinical samples used in the present disclosure were provided by the Affiliated Hospital of Xi'an Jiaotong University. Patients were informed of the information and the samples were approved by the ethics committee.

Example 4 shows the data analysis of an RA mouse model.

Protein contents of pyroglutamyl aminopeptidase in sample serums in Examples 1-3 were carried out through a Human PGPEP1 (Pyroglutamyl-peptidase 1) enzyme-linked immunosorbent assay (ELISA) Kit. A protein content pyroglutamyl aminopeptidase in sample serums in Example 4 was carried out through a Mouse Pyroglutamyl-peptidase 1 (PGPEP1) ELISA Kit. All sample serums used in the ELISA tests were prepared according to a standard scheme. The sample serums were diluted, and detection procedure was strictly carried out according to commercial ELISA kit protocol.

### Example 1

Protein contents of pyroglutamyl aminopeptidase in serums of 10 healthy volunteers and 10 RA patients (who are diagnosed without considering the positive or negative status of RF and CCP) were detected as shown in Table 1. A receiver operating characteristic (ROC) curve was analyzed to identify indicators with high area under the curve (AUC), sensitivity, and specificity, thereby determining an optimal screening positive cutoff value. The closer the AUC is to 1, the better the diagnostic effect is.

TABLE 1
Content of Pyroglutamyl Aminopeptidase in Serum Samples (Upper
Arm Venous Blood) of the RA Patients and Healthy Volunteers

| | Content of Pyroglutamyl Aminopeptidase in the Serum Samples (ng/mL) | | | | | | | | | |
|---|---|---|---|---|---|---|---|---|---|---|
| RA | 125.26 | 112.68 | 60.27 | 60.27 | 84.38 | 74.95 | 83.86 | 60.79 | 59.75 | 66.56 |
| samples | | | | | | | | | | |
| Healthy | 26.44 | 28.05 | 22.00 | 26.03 | 31.27 | 26.84 | 27.64 | 28.45 | 28.45 | 24.42 |
| samples | | | | | | | | | | |

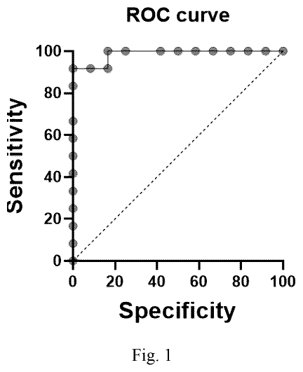

Referring to FIG. 1, the result of AUC=0.986, p<0.0001 showed that the content of pyroglutamyl aminopeptidase can be used to clearly distinguish RA patients from healthy volunteers. When a cut-off value was 57.28, the specificity for judging RA was 100% and the sensitivity is 91.67%, that was, when this cut-off value was taken, subjects without RA can be identified 100%, and false positivity would not occur. A simultaneous detection rate (or true positive rate) was 91.67%. The high sensitivity can effectively identify RA patients and reduce the rate of missed diagnosis. In other words, when the content of pyroglutamyl aminopeptidase was greater than 57.28, the subject can be preliminarily identified as suffering from RA.

### Example 2

In this example, 4 RA patients and 4 osteoarthritis (OA) patients have similar external symptoms, including morning stiffness, hypertrophy of finger joint tissues, and a sensation of bone friction. It was difficult to effectively differentiate them merely through external joint manifestations. In this example, the RA patients and the OA patients can be effectively differentiated through detecting the content of pyroglutamyl aminopeptidase in venous blood of these patients considering the cut-off value of 57.28 ng/mL, and such tests were carried out through the method the same as that in Example 1.

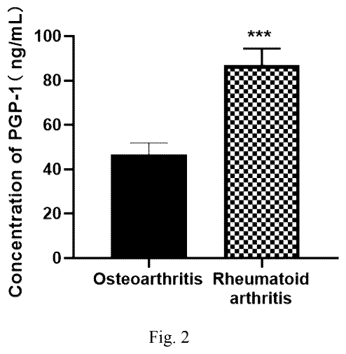

Test results were shown in FIG. 2, the content of pyroglutamyl aminopeptidase in serums of OA patients was 46.6 ng/mL, which was lower than the cut-off value of 57.28 ng/mL, while the content of pyroglutamyl aminopeptidase in serums of the RA patients was 86.9 ng/ml, which was much higher than the cut-off value. Such data showed that pyroglutamyl aminopeptidase can effectively differentiate RA and OA with P<0.001.

### Example 3

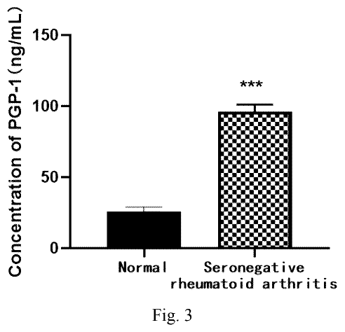

In this example, blood samples of 3 RA patients with seronegative rheumatoid arthritis (RF-negative and CCP-negative) were collected from RA patients in the hospital, including 2 females and 1 male, with an average age of 55 years and an average disease duration of 2 years. Three normal blood samples were collected from the healthy volunteers, and detected according to the method in Example 1, and the results were shown in FIG. 3.

Referring to FIG. 3, the pyroglutamyl aminopeptidase in the RA patients with seronegative rheumatoid arthritis was still highly expressed at 96±5 ng/mL, which was much higher than the cut-off value of 57.28 ng/ml, P<0.001, indicating that pyroglutamyl aminopeptidase can be used for the diagnosis of seronegative rheumatoid arthritis.

### Example 4

An RA mouse model including 12 male DBA/1 mice was constructed. Lipopolysaccharide and Freund's adjuvant were completely emulsified to obtain a emulsified solution. The emulsified solution was administered intradermally at a dose of 0.2 mg on day 0 and day 7. Generally, an inflammatory response of joint would occur in 10-13 days. A thickness of a paw of each mouse was measured using a vernier caliper with 0.01 mm precision, and clinical scoring was performed based on two parameters as shown in Table 2: inflammation severity in a toe and swelling degree of the paw.

TABLE 2
Inflammatory Response of Paw Joint of each Mouse

| | Score | | Score |
|---|---|---|---|
| Per Mouse | (0-2) | Per Paw | (0-3) |
| No inflammatory toe | 0 | Paw thickness increased ≤30% | 0 |
| | | compared with a control | |
| 1-5 inflammatory toes | 0.5 | Paw thickness increased ≥30% | 1 |
| | | compared with a control | |
| 5-10 inflammatory | 1 | Paw thickness increased ≥50% | 2 |
| toes | | compared with a control | |
| 11-15 inflammatory | 1.5 | Paw thickness increased ≥80% | 3 |
| toes | | compared with a control | |
| >15 inflammatory | 2 | | |
| toes | | | |

The score of each mouse was the sum of inflammation score of the toe and the swelling score of the paw, where a maximum was 14 points. The score of 1.5 or above was considered a successful model construction. These RA model-constructed mice were then randomly re-grouped and continued with subsequent administration experiments.

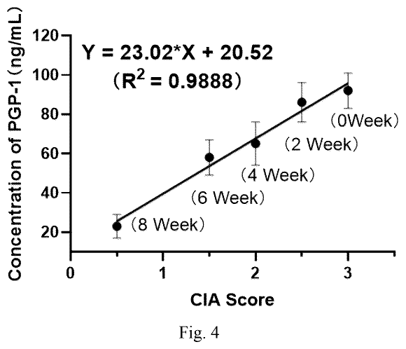

Ten mice with successful modeling with 3 points scores were selected to continue the experiment. The experiment was divided into a model control group and a drug administration group, with 5 mice in each group. The mice in the model control group received no treatment, while the mice in the drug administration group were orally administered methotrexate via gavage at t a dose of 14 mg/kg/week once a day for three weeks. Methotrexate was a cornerstone drug for the treatment of rheumatoid arthritis. 2018 Chinese Guidelines for the Diagnosis and Treatment of Rheumatoid Arthritis recommended a single use of methotrexate or as a cornerstone drug for combination therapy. After the treatment began, CIA scores were carried out to each mouse every two weeks, and blood was collected from a tail vein to determine the content of pyroglutamyl aminopeptidase in blood through a Mouse Pyroglutamyl-peptidase 1 (PGPEP1) ELISA Kit. The results showed that the CIA score and the content of pyroglutamyl aminopeptidase basically unchanged, where the CIA score was about 3.0, and an average content of pyroglutamyl aminopeptidase was about 90 ng/ml. In addition, the drug administration group was plotted with the CIA score as an abscissa and the content of pyroglutamyl aminopeptidase as an ordinate. Results were shown in FIG. 4. As the CIA score of the drug administration group gradually decreased, the content of pyroglutamyl aminopeptidase also gradually decreased. A regression equation was Y=23.02X+20.52, with a goodness of fit of R²=0.9888.

After the RA model-constructed mice received methotrexate treatment, the concentration of pyroglutamate aminopeptidase 1 (PGP-1) in their serums gradually decreased with the treatment time, and its downward trend was consistent with the improvement trend of the inflammatory score, which indicates that PGP-1 can be used to reflect the disease progression during the treatment process and has potential prognostic assessment value.

In conclusion, the above experimental data indicated that the level of pyroglutamyl aminopeptidase was positively correlated with the progression degree of the RA, suggesting that the level of pyroglutamyl aminopeptidase in serum can be used for classification of the RA and prognostic assessment.

Described above are detailed description of the present disclosure, which are not intended to limit the disclosure. It should be understood that various modifications and replacements made by those of ordinary skill in the art without departing from the spirit of the present disclosure shall fall within the scope of this application defined by the appended claims.

What is claimed is:

1. An application of a reagent for quantitative detection of pyroglutamyl aminopeptidase in the preparation of a product for diagnosis of rheumatoid arthritis or a product for prognostic assessment of rheumatoid arthritis.

2. The application of claim 1, wherein the rheumatoid arthritis is selected from the group consisting of rheumatoid factor-positive (RF-positive) rheumatoid arthritis, anti-cyclic citrullinated peptide antibody-positive (CCP-positive) rheumatoid arthritis, RF-negative and CCP-negative rheumatoid arthritis, and a combination thereof.

3. The application of claim 1, wherein the reagent for quantitative detection of pyroglutamyl aminopeptidase is a reagent for quantitative detection of pyroglutamyl aminopeptidase gene mRNA, a reagent for quantitative detection of pyroglutamyl aminopeptidase protein or a combination thereof.

4. The application of claim 1, wherein the quantitative detection is performed by enzyme-linked immunosorbent assay (ELISA), Western blot, immunohistochemistry, a quantitative fluorescent polymerase chain reaction, mass spectrometry, or a combination thereof.

5. The application of claim 1, wherein the product for diagnosis of rheumatoid arthritis or the product for prognostic assessment of rheumatoid arthritis is a kit, a chip, a test strip, a high-throughput sequencing platform or a combination thereof.

6. The application of claim 1, wherein the product for diagnosis of rheumatoid arthritis is adapted to diagnosis of rheumatoid arthritis in chronic joint diseases comprising rheumatic arthritis and osteoarthritis, classification diagnosis of rheumatoid arthritis or a combination thereof.

7. A detection system for diagnosis of rheumatoid arthritis, comprising:
a sample collection module;
a sample detection module; and
a data analysis module;
wherein the sample collection module is configured to collect a biological sample from a subject;
the sample detection module is configured to quantitatively detect an expression level of pyroglutamyl aminopeptidase in vitro; and
the data analysis module is configured to compare a detection result with a diagnostic threshold, and generate a diagnosis report.

8. The detection system of claim 7, wherein the biological sample is selected from the group consisting of whole blood, serum, plasma, tissue fluid and a combination thereof.

9. A product for diagnosis or prognostic assessment of rheumatoid arthritis, comprising:
a reagent for detecting an expression level of pyroglutamyl aminopeptidase in a sample.

10. The product of claim 9, wherein the product is selected from the group consisting of an enzyme-linked immunosorbent assay (ELISA) kit, a Western blot kit, an immunohistochemistry staining kit, an immunochromatographic test strip and a combination thereof.
